(19) United States
(12) Patent Application Publication
(10) Pub. No.: US 2010/0140500 A1
(43) Pub. Date: Jun. 10, 2010

(54) APPARATUS AND METHOD FOR THE TREATMENT OF BREAST CANCER WITH PARTICLE BEAMS

(75) Inventors: Jerry M. Jesseph, Bloomington, IN (US); Frederick B. Jesseph, Bloomington, IN (US)

Correspondence Address: SHAY GLENN LLP, 2755 CAMPUS DRIVE, SUITE 210, SAN MATEO, CA, 94403, US

(73) Assignee: THERAPY POSITIONING TECHNOLOGIES, LLC, New York, NY (US)

(21) Appl. No.: 12/664,970

(22) PCT Filed: Jun. 19, 2008
(86) PCT No.: PCT/US08/07627

§ 371 Date: Jan. 26, 2010

Related U.S. Application Data
(60) Provisional application No. 60/936,253, filed on Jun. 19, 2007.

(51) Int. Cl. A61N 5/00; A61B 8/14

(52) U.S. Cl. 250/454.11; 600/443; 250/492.3

(57) ABSTRACT

An apparatus and method for treating a tumor mass location in a breast of a patient. The breast is stabilized in space, preferably by creating a reduced pressure environment around the breast. Particle beams are applied to a tumor mass location positioned at the center of a substantially spherical (or cylindrical) portion of a chamber filled with a fluid having an electron density substantially similar to the breast. The patient treatment platform is translated to aim the particle beam at the tumor mass location while traversing and beaming perpendicular to the portion of the chamber.

### CROSS-REFERENCE TO RELATED APPLICATIONS

This application claims priority to and the benefit of U.S. Provisional Patent Application No. 60/936,253 entitled “DEVICE AND METHOD FOR THE TREATMENT OF BREAST CANCER WITH PARTICLE BEAMS” filed 19 Jun. 2007.

### BACKGROUND OF THE INVENTION

Breast cancer remains a scourge on humanity. For early-stage breast cancers progress has been made toward improved local control and better cosmetic outcomes. However, present-day approaches require multiple radiographic techniques and interventions just to establish a diagnosis of breast cancer. In addition, an estimate must be made of the extent of, and possible metastatic spread of the cancer, even before proceeding with treatment. This may take many weeks to coordinate and accomplish.

The treatment phase for breast cancer again requires multiple procedures, high cost and delays. The entire process can extend over many weeks. It typically requires anesthesia, surgery, lymphatic tracing and sampling, along with post operative radiation therapy of part, or all, of the breast. Cosmetic results vary widely and the cost is great.

### SUMMARY OF THE INVENTION

One embodiment of the present invention is a method of treating a tumor mass location in a breast of a patient, including directing an isocenter of a particle beam at a center of a spherical portion of an outer chamber that overlaps the tumor mass location after passing the particle beam through a first fluid at least partially filling the outer chamber. Next it passes the beam through a second fluid at least partially filling an inner chamber having a reduced pressure environment that encompasses at least a portion of the breast.

In one refinement of the embodiment the particle beam is a proton particle beam, and the first fluid and the second fluid are the same and have an electron density substantially similar to the breast.

Another refinement of the embodiment includes rotating the particle beam and the breast relative to one another while continuing to direct the isocenter of the particle beam at the center of the spherical portion of the outer chamber.

Another refinement of the embodiment includes creating the relative rotation by moving a robotic arm connected to a treatment platform upon which the patient lays.

Another refinement of the embodiment includes deactivating the particle beam at various times while rotating the particle beam and the breast relative to one another.

In another refinement of the embodiment the particle beaming occurs after a surgical intervention on the breast, and further comprising deactivating the particle beam when the particle beam would otherwise pass through an existing incision site on the breast.

Another refinement of the embodiment includes deactivating the particle beam when the beam would otherwise pass through a planned future incision site on the breast.

Another refinement of the embodiment includes directing the particle beam so that it only passes through an underside of the breast of the patient.

Another refinement of the embodiment includes first imaging the breast and obtaining data regarding the tumor mass location within the breast and providing the data to a computer directing the particle beams at the breast.

In another refinement of the embodiment the imaging is done using a combination of 3-D ultrasound and CT.

Another embodiment of the present invention is a method of treating a tumor mass location of a breast, including directing an isocenter of a particle beam toward a center of a spherical portion of a chamber enclosing the breast and through a fluid having an electron density substantially similar to the breast. The center of the spherical portion of the chamber overlaps the tumor mass location, and the particle beam is substantially normal to the spherical portion of the chamber.

One refinement of the embodiment includes rotating the breast and spherical portion of the chamber relative to the particle beam to scan through a plurality of particle beam entry angles into the breast while continuing to direct the particle beam substantially normal to the spherical portion of the chamber.

Another refinement of the embodiment includes directing the particle beam only through an underside of the breast.

Another refinement of the embodiment includes deactivating the particle beam except when scanning an underside of the breast.

Another refinement of the embodiment includes rotating the breast and spherical portion of the chamber relative to the particle beam by moving a robotic arm connected to a treatment platform upon which the patient lays.

Another refinement of the embodiment includes deactivating the particle beam at various times while rotating the particle beam and the breast relative to one another.

Another refinement of the embodiment includes first imaging the breast and obtaining data regarding the tumor mass location within the breast and providing the data to a computer directing the particle beam at the breast.

In another refinement of the embodiment the imaging is done using a combination of 3-D ultrasound and CT.

Another embodiment of the present invention is a method of treating a tumor mass location of a breast of a patient, including reducing pressure within an inner chamber that encloses at least a portion of the breast after creating a fluid tight seal between the inner chamber and the patient. The inner chamber is at least partially filled with a fluid having an electron density substantially similar to the breast, and positions an outer chamber at least partially filled with the fluid so that a center of a spherically shaped portion of the outer chamber overlaps the tumor mass location. A particle beam is scanned across an arc of a portion of the outer chamber while directing an isocenter of a particle beam at the center of the portion.

One refinement of the embodiment includes scanning the particle beam only across an underside of the breast.

Another embodiment of the present invention is a method of treating a breast cancer tumor mass location, including stabilizing the breast, and applying an isocenter of a particle beam to a center of spherical portion of an at least partially fluid filled chamber that encloses at least a portion of the breast. The fluid has an electron density substantially similar to the breast, and the center overlaps the tumor mass location.

In one refinement of the embodiment the breast is stabilized by providing a reduced pressure environment around the breast.

Another refinement of the embodiment includes providing the reduced pressure environment using a fluid tight seal between a wall defining an inner chamber and a patient's body, and positioning the hemispherical portion of the chamber around at least a portion of the breast.

Another refinement of the embodiment includes first imaging the breast to determine data regarding the tumor mass location within the breast, and providing the data to a computer controlling the application of the particle beam.

Another embodiment of the present invention is a method of treating a tumor mass location in a breast of a patient, including stabilizing the breast within a reduced pressure environment enclosed by a chamber having a wall at least a portion of which is spherically shaped; imaging the breast to determine the location of the tumor; beaming the tumor with particles through the spherically shaped portion of the chamber; and rotating the beam relative to the tumor mass location around a center of the spherically shaped portion of the chamber, wherein the center overlaps the tumor mass location.

In another refinement of the embodiment the patient is prone on a treatment platform, and the patient is rotated with respect to at least one stationary particle beam.

Another refinement of the embodiment includes directing the particle beam only through an underside of the breast.

Another refinement of the embodiment includes deactivating the particle beam except when scanning an underside of the breast.

Another refinement of the embodiment includes using fiducial markers positioned within or on the breast to identify the tumor mass location.

In another refinement of the embodiment, during stabilization of the breast and during beaming the tumor with particles, the chamber is filled with a fluid having an electron density substantially similar to the breast.

In another refinement of the embodiment the beaming is controlled to overlap a biopsy track along which prior surgical intervention of the breast has occurred.

Another embodiment of the present invention is an apparatus for treating a tumor location in a breast, including a first wall having an interior surface defining a first chamber sized to encompass at least a portion of the breast. The apparatus includes means for stabilizing the breast within the first chamber. This apparatus also includes a second wall having an internal surface defining a second chamber, the second wall including a spherical (or cylindrical) portion around a center. The first chamber is smaller than and positioned substantially within the second chamber, and the second chamber is movable with respect to the first chamber to position the center of the spherical portion to overlap the tumor location.

In one refinement of the embodiment an edge of the first wall includes an adhesive for attaching the first wall to the breast in a substantially fluid tight fashion.

In another refinement of the embodiment the adhesive is a replaceable tape.

In another refinement of the embodiment the spherical portion of the second wall is substantially hemispherically shaped.

In another refinement of the embodiment at least a portion of the first wall is substantially hemispherically shaped.

In another refinement of the embodiment the spherical portion of the second wall is substantially hemispherically shaped.

In another refinement of the embodiment at least a portion of the first wall is visually transparent.

Another refinement of the embodiment includes a fat equivalent fluid at least partially filling the first chamber and the second chamber.

In another refinement of the embodiment the means for stabilizing the breast within the first chamber is a reduced pressure environment generated by a pump fluidly connected to the first chamber by a conduit connected to a first port in the first wall, and wherein the conduit passes through a second port in the second wall.

In another refinement of the embodiment the first wall defines a first port and further includes a valve within the first port.

In another refinement of the embodiment the first and/or second wall is manufactured from a material that may be sterilized for reuse.

In another refinement of the embodiment the first and/or second wall is a plastic.

In another refinement of the embodiment the first and/or second wall is a composite.

Another embodiment of the present invention is an apparatus for treating a tumor location in a breast including a first shell having an interior surface defining a first chamber sized to encompass at least a portion of the breast. At least a portion of the shell defines a spherical portion having a center. At least a portion of the first chamber is filled with a fluid having an electron density substantially similar to breast tissue. The apparatus further includes means for stabilizing the breast within the first shell.

In another refinement of the embodiment the portion of the first shell is substantially hemispherically shaped.

In another refinement of the embodiment an edge of the shell includes an adhesive for attaching the shell to the breast in a substantially fluid tight fashion.

In another refinement of the embodiment the adhesive is a replaceable tape.

In another refinement of the embodiment an edge of the shell includes molding shaped to overlay an area around the breast.

Another refinement of the embodiment includes a second shell having an internal surface defining a second chamber. The second shell includes a spherical portion around a second center, and the first chamber is smaller than and positioned substantially within the second chamber. The second chamber is movable with respect to the first chamber to position the second center to overlap the tumor location.

Another refinement of the embodiment includes the fluid at least partially filling the second chamber.

In another refinement of the embodiment the means for stabilizing the breast within the first chamber is a reduced pressure environment generated by a pump fluidly connected to the first chamber by a conduit connected to a first port in the first shell. The conduit passes through a second port in the second shell.

In another refinement of the embodiment the shell defines a port.

Another refinement of the embodiment includes a valve in the port of the shell.

In another refinement of the embodiment the first and/or second wall is manufactured from a material that may be sterilized for reuse.

In another refinement of the embodiment the first and/or second wall is a plastic.

In another refinement of the embodiment the first and/or second wall is a composite.

### BRIEF DESCRIPTION OF THE FIGURES

FIG. 1 illustrates a side cross-sectional view of one embodiment of a breast fixation apparatus encompassing at least a portion of a breast having a tumor.

FIG. 2 illustrates a side cross-sectional view of the embodiment of a breast fixation apparatus of FIG. 1 in which the spherical portion of the fixation apparatus is centered on a tumor mass location in a different location within the breast.

FIG. 3 illustrates a top view of a fixation apparatus of the present invention enclosing a breast with a tumor.

FIG. 4 illustrates a side view of a treatment apparatus of the present invention.

FIG. 5 illustrates a side cross-sectional view of an inner chamber of a multi-chamber fixation and treatment apparatus of the present invention.

FIG. 6 illustrates a side cross-sectional view of the inner chamber and outer chamber of a multi-chamber fixation and treatment apparatus of the present invention.

### DETAILED DESCRIPTION OF THE PREFERRED EMBODIMENTS

For purposes of promoting an understanding of the principles of the invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated herein being contemplated as would normally occur to one skilled in the art to which the invention relates.

As previously noted, the current standard of care for treatment of breast cancer includes anesthesia, surgery, lymphatic tracing and sampling, along with post operative radiation therapy of part, or all, of the breast. Thus, at present, a patient who develops breast cancer has no choice but to accept scarring and deformity of the breast—or to have the breast removed entirely. The present invention is a novel method and apparatus to treat breast cancers (particularly early-stage breast cancers) using protons, or other heavy ion particle beams. As used herein, the term “particle beam” includes those particle beams that have undergone testing and/or clinical evaluation such as protons, carbon, pions or pi mesons, helium (alpha particles), neon, iron and even anti-matter in the form of anti-protons.

Particle beam radiotherapy has shown superior dose distribution in tissues, and provides new opportunities for the treatment of breast cancer. Partial breast radiation has shown increasing promise in the treatment of early stage cancer, thus particle therapy appears to be a feasible approach. Many variations in technique along with different fractionation schedules are possible. The approach of the present invention could provide for shorter treatment times and better cosmetic results, and could prove to be more cost effective as well. One challenge for radiation of the breast has been reproducibly immobilizing the breast for treatment planning and proper targeting. In addition, high skin dosage represents another challenge. Various embodiments of the present invention provide for improved treatment options.

The methods and apparatus of the present application are preferably used in conjunction with a proton or carbon particle beam. While other particle beams might at present hint at better clinical results (such as anti-protons), cost constraints in the production or use of other particle beams might preclude their widespread usage. Results from proton and carbon beam treatments for cancers, such as ophthalmic melanomas, head and neck tumors, chordomas, and, recently, prostate and lung cancers are very encouraging. Particle beam treatments have the ability to provide tumoricidal dosage within tissues with millimeter geometric accuracy along with precision dosage accuracy—while sparing surrounding normal tissues. Given existing economic tradeoffs, proton beams are presently preferred. However, the usage of other particle beams is contemplated as within the scope of the invention unless specifically claimed otherwise herein.

Recent developments in breast conserving therapies have validated a number of principles. By removing the tumor mass along with a rim of normal tissue, and including the tumor bed and all or part of the breast in the radiation field—local control similar to that with mastectomy is often achieved. Conserving the breast has many physical and psychological advantages. However, present multi-modality approaches often take a long time to accomplish and are very expensive. Other ablative techniques (examples include RF ablation, and hypothermia or hyperthermia probes) have proven inadequate for local control.

Image acquisition and/or treatment planning preferably include stabilization of the breast. In one embodiment of the present invention, such stabilization of the breast is accomplished using a means for stabilizing the breast, such as a breast fixation apparatus. Examples of such a breast fixation apparatus are described in U.S. Pat. No. 6,254,614, entitled “DEVICE AND METHOD FOR IMPROVED DIAGNOSIS AND TREATMENT OF CANCER,” that issued on 3 Jul. 2001, the contents of which are incorporated herein by reference. While other apparatus and methods for stabilizing the breast are contemplated as within the scope of the invention (as further discussed below), the various embodiments of the present invention described and illustrated herein will focus on this preferred mechanism. Namely, the preferred breast fixation apparatus stabilizes the breast in space by providing a reduced pressure environment around the breast. Stabilizing the breast in space allows reproducible positioning, and permits determination and recordation of various data sets concerning, for example, tumor mass location, using imaging techniques. In one embodiment, imaging is preferably done using a combination of CT and 3-D ultrasound. This allows for precise fusion of data with options for better clarification of tissue characteristics.

The current standard of care involves surgical intervention (with associated anesthesia, incision site(s), etc.) with post-operative radiation therapy. As discussed below, in view of the current standard of care it is assumed that early applications will be those in which the radiation therapy is either supplemented or replaced by particle beam therapy according to the methods and apparatus of the present invention. That is to say, a tumor is at least partially, but preferably entirely, surgically excised from the breast followed by particle beam therapy. Additionally, the methods and apparatus of the present invention might find use as a pre-operative form of therapy.

Unlike photon treatment (such as x-rays and gamma rays) some particle beams penetrate a known distance into tissue. Therefore, they are often used to treat cancers located on the surface of or below the skin. As an example, in proton particle beam therapy, protons deposit their energy over a very small area, which is called the Bragg peak. The Bragg peak can be used to target high doses of proton beam therapy to a tumor while doing less damage to normal tissues in front of and behind the tumor. Thus, it is contemplated as within the scope of the invention that clinical results might eventually result in evolution of the standard of care to permit omission of surgical intervention, and treatment of breast cancers, particularly early stage breast cancers, solely by particle beaming. That is to say, clinical success might be achieved with particle beam therapy alone, and obviate the need for surgery. Consequently, it is contemplated as within the scope of the invention that applications of one or more embodiments of the present invention will preferably eventually be used without the need for anesthesia, surgery and/or post-operative (internal or external) radiation treatments. It should be understood, however, that it is also contemplated as within the scope of the invention that the methods and apparatus of the present invention might be used in conjunction with anesthesia, surgery or post-operative radiation. However, various applications of at least some embodiments of the present invention present the possibility of treating early-stage breast cancer more cheaply—possibly in a single session—leaving minimal scarring (or no scar at all) on the exterior of the breast.

Various embodiments of the present invention are contemplated for use with or without surgical intervention. Thus, as used herein, the term “tumor mass location” is intended to be construed broadly. It should be understood that the term tumor mass location might refer to the location of an existing tumor mass within the body. However, it should further be understood that as used herein a tumor mass location is present even in those instances in which particle beam therapy is preceded by surgical intervention in which the tumor mass is partially and/or entirely excised. Even after, for example, a lumpectomy removal of cells, the location of the resultant cavity (that fills with fluid and shrinks and closes after the surgery) is considered the tumor mass location. Similarly, when referring to or claiming herein treatment of a breast cancer tumor, it is contemplated as within the scope of the invention that such refers to treatment of the location of an existing tumor or to the location at which a tumor was thought present prior to surgical intervention.

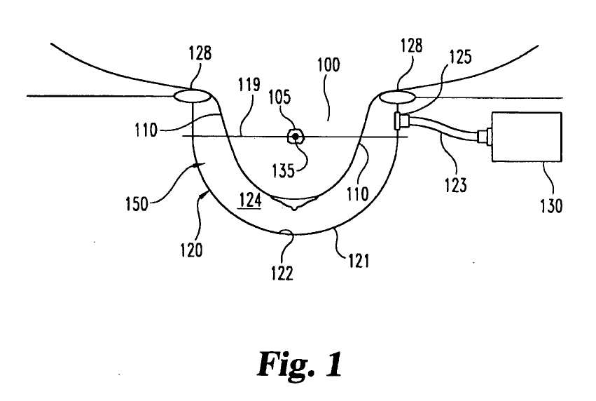

With reference to FIG. 1, there are illustrated aspects of one embodiment of the present invention. The exterior surface 110 of a breast 100 of a patient having a tumor mass location 105 requiring treatment is placed at least partially (and, as illustrated in FIG. 1, preferably entirely) within a fixation apparatus 120. The breast fixation apparatus 120 preferably includes at least one port 125 connected to a pump 130 by a conduit 123. The conduit 123 or the breast fixation apparatus 120 preferably includes a valve at port 125. The presence of such a valve in conjunction with use of a conduit 123 that is detachable from the port 125 permits the pump 125 to reduce the pressure within, and then the valve is closed and conduit 123 can be removed. Thus, the apparatus still attached to the patient during imaging and/or interventional procedures is minimized. Aspects such as a closable valve and removable conduit (and pump) are preferable, but not necessary, in this embodiment (as well as embodiments discussed below that further include a second outer chamber that encompasses at least a portion of the inner chamber). As will be discussed below, only the inner chamber need include a reduced pressure environment. Additionally, fixation apparatus 120 might be pressed against the body by a strap or band (not illustrated) that extends around the patient to be tied, or tightened similar to a belt. Alternatively, the fixation apparatus might be constructed as part of a bra-like apparatus for retention on the patient's chest and breast(s).

In one embodiment, the cup-like fixation apparatus 120 is preferably at least partially spherical (the portion of apparatus 120 in FIG. 1 that lies below the imaginary line 119 that passes through the center 135 of the spherical portion). In one refinement of such an embodiment, the fixation apparatus 120 preferably includes a substantially rigid plastic spherical portion with an outer surface 121 and an inner surface 122. The plastic (or composite) is preferably substantially transparent to the particular imaging modality (or modalities) used. An internal cavity 124 exists between the inner surface 122 of fixation device and the exterior surface 110 of the breast 100. The cavity 124 is preferably filled with fluid 150. Aspects of fluid 150 are discussed further below.

As illustrated in FIG. 1, the center 135 of the spherical portion of fixation apparatus 120 is preferably at the center, or substantially overlaps the center, of the tumor mass location 105. It should be understood that a tumor mass location is not necessarily a symmetrical volume, and thus the concept of a center for the tumor mass location is broader than the typical geometrical concept of a center of a circular area or spherical volume. The center of the tumor mass location will be selected to maximize the energy dose delivered by the incident particle beam to potentially cancerous cells while minimizing the dose received by healthy surrounding tissue. The energy dose resulting from the particle beam, however, is typically modeled as a spherical volume with smaller dosages received the further the distance from the isocenter of the particle beam. It should further be understood that an oddly shaped tumor mass location might be treated as having more than one center to subject to treatment by particle beam therapy. In other words, multiple overlapping spheres of smaller radius resulting from the particle beam energy dose might be preferred to a single sphere of a larger radius.

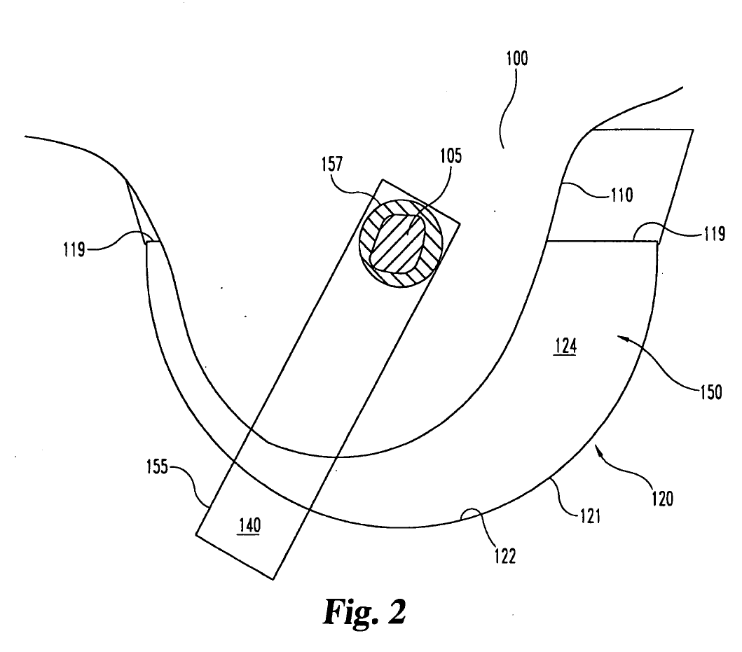

With reference to FIG. 2, wherein like elements are labeled as previously described, a particle beam 140 is directed at the tumor mass location 105 along a single trajectory designated by the lines making up rectangle 155. Particle beam 140 is aimed at the center 135 (see FIG. 1) of the spherical portion of fixation apparatus 120, and the particles are intended to have a penetration depth that extends to that center with a maximum energy dose release in a substantially spherical volume 157 within breast 100. Particles that travel a trajectory normal to the surface of the spherical portion of the fixation apparatus 120 have an equal path of electron density to the center of the hemisphere. This is because the spherical volume traversed by the particle beam preferably includes either breast tissue or fluid 150. The fluid 150 acts as a compensator to allow delivery of spread-out Bragg peak energies precisely to the tumor mass location 105 and to a precise margin (volume 157 of FIG. 2) of the surrounding tissues. In one refinement of this embodiment, this is accomplished by placing the tumor center (preferably tagged with a fiducial marker during surgery) at the center of the sphere created by the fixation device. The fixation apparatus retains within it the electron-isodense fluid medium and the breast tissue itself. By creating an “anatomy of advantage,” it is possible to deliver uniform dosage—due to substantially identical path radii to the center of the spherical portion. Fluid 150 is preferably a fat equivalent fluid (such as water) that has an electron density substantially similar to that of the breast. As used herein, fluid is intended to encompass both liquids and gels, it being understood that a gel is contemplated as within the scope of the invention for filling the interior volume of the chamber (or chambers as in below described embodiments) surrounding the breast.

The fluid 150 within volume 124 of fixation apparatus 120 is preferably held at a slightly reduced pressure to expand the breast 100 to its structural envelope. It should be understood that fluid 150 only needs to be present in the portion of volume 124 that is below the imaginary line 119 that partially delineates the spherical portion of fixation apparatus. However, fluid 150 might fill the entire cavity between the exterior surface 110 of breast 100 (and the chest wall) and internal surface 122 of fixation apparatus 120. Air is pumped out of the internal cavity 124 through port 125 by pump 130. The fluid 150 preferably replaces the air as it is pumped out, but might be pumped in before or after pumping out the air. For patient comfort, the fluid 150 is preferably first heated to approximately body temperature.

Creating the reduced pressure environment requires a substantially fluid tight seal between the rim of the fixation apparatus 120 and the patient's body. As illustrated in FIG. 1, the rim might include patient specific molding 128 that is shaped to conform to the chest of the patient's body, and to provide a fluid tight seal. It should be understood, however, that it is contemplated as within the scope of the invention that the rim of the fixation apparatus may be attached entirely to the chest, entirely to the breast, or to some combination of the chest and breast.

Once a tissue diagnosis is established, fiducial markers are positioned to identify a location relative to, and preferably substantially at or near to the center of the tumor mass location. An example includes, but is not limited to, clips left in place during surgical excision of the tumor mass. The fiducial markers might be positioned within the breast at the tumor mass location. Alternatively, the fiducial markers for identifying the tumor mass location might be positioned on the skin of the breast. Treatment planning with a particle beam also includes determination of the isocenter of treatment and the dose. As used herein, the term isocenter is the position at which there is a maximum of the energy dose delivered by the particle beam.

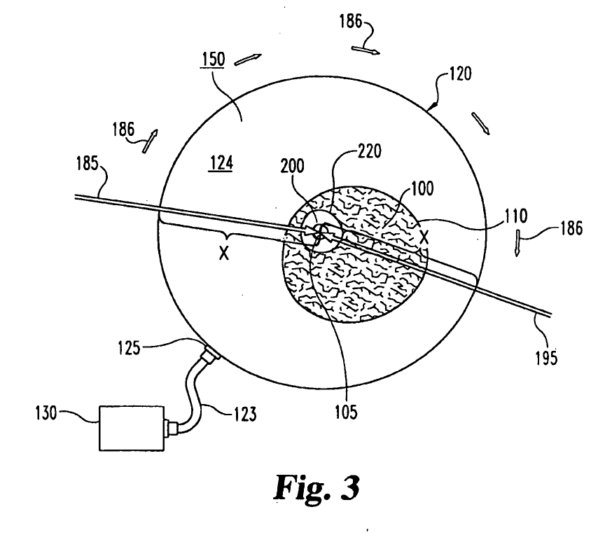

With reference to FIG. 3, wherein like reference numerals are used as in FIG. 1, the isocenter of the treatment preferably is at the center of the tumor mass location 105. The breast 100 is placed in a reduced pressure environment within fixation apparatus 120. The reduced pressure environment is preferably filled with a fat equivalent fluid 150. In one application a first particle beam 185 is applied with an isocenter that overlaps the tumor mass location 105. The patient is then rotated (as indicated by the arrows 186), with the beam off, and a second particle beam 195 is applied having an isocenter that overlaps the tumor mass location 105, preferably at the same position as the first particle beam. Both the particle beam 185 and the particle beam 195 travel substantially identical path radii (labeled as distance X in FIG. 3) to the center of the spherical. Alternatively, the particle beam could be on the entire time during patient rotation and scan across the surface of the breast while being aimed or directed (having an isocenter) at the tumor mass location 105. The dose will preferably include a tumoricidal sphere 200 around the tumor 105, and a larger sphere 220 of lesser dosage.

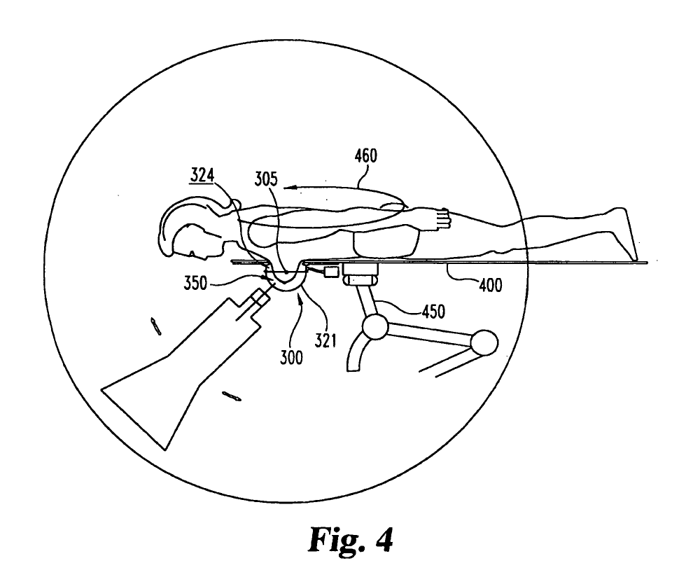

As illustrated in FIG. 4, in one embodiment treatment is provided to the breast using the breast fixation apparatus. The patient is preferably in a prone position, so that the isocenter 305 overlaps the center of a spherical portion 300 projecting below the movable treatment platform 400. The cavity 324 within the spherical portion will be filled with a fluid 350 whose electron density is similar or identical to that of the breast tissue. The same reduced pressure environment preferably exists within cavity 324 as used for imaging and/or treatment planning. This provides a compensator for the path of the particle beam, which, in fact, converts any size and shape of breast into a substantially spherical shape along a range of potential trajectories. Such a compensator allows more straightforward beam energy calculations and equidistance to the tumor mass location from any point on the outer surface 321 of the breast fixation apparatus 320.

With the treatment plan determined, and the tumor securely placed at the center of the spherical portion of the breast fixation apparatus, treatment preferably proceeds with a low-angle gantry trajectory aimed at the isocenter. In this position, using a robotic arm 450 controlling the treatment platform 400, the patient will be rotated (as indicated by arrow 460) around the isocenter with the beam on. This will smear the skin dosage around the breast and allow for simple changes of gantry angle to allow multiple arcs of treatment. Due to the forced symmetry with the fluid compensator, the patient may be spun about the isocenter (that overlaps the tumor mass location) preferably with a beam of constant energy. The gantry angle may be changed and the same patient motion from before may be used. However, it should be understood that tumors of irregular shape might preferably be subject to beam energies of greater energy along select entry angles into the breast. Also, tumors of irregular shape might instead, or in addition, require beaming more than one center of a tumor mass location.

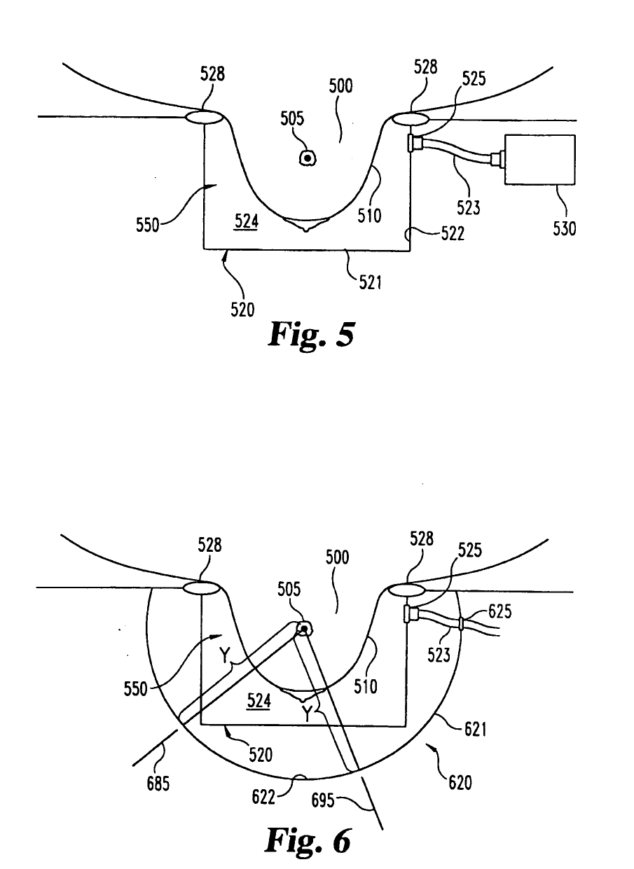

One embodiment of the apparatus allows for improved placement of the tumor at the center of a “sphere” using two chambers as illustrated in FIGS. 5-6. The inner chamber of the device is filled with electron isodense fluid and provides negative pressure to stabilize the breast. The larger outer chamber is preferably filled with the same isodense fluid. The outer chamber can be moved around the smaller inner chamber so the tumor can be centered within the larger (outer) chamber, without making adjustments in the position of the tumor—without moving the breast (or the patient). Thus, FIGS. 5-6 illustrate aspects of a dual chamber apparatus for fixation and treatment of a breast. FIG. 5 illustrates an inner chamber 520 of an embodiment that could allow for improved placement of the tumor at the center of the outer chamber 620. Briefly, the breast 500 is enclosed within an inner chamber 520. Inner chamber 520 may be hemispherical or other shape. As illustrated in FIG. 5, inner chamber 520 is a cylindrical chamber. Outer chamber 620 (see FIG. 6) is positioned so that the tumor 505 is at the center of the larger outer chamber 620 around the inner chamber 520, the latter being fixed to the patient.

With reference to FIG. 5, the exterior surface 510 of a breast 500 of a patient having a tumor mass location 505 requiring treatment is placed at least partially (and, as illustrated in FIG. 5, preferably entirely) within a fixation apparatus 520. The breast fixation apparatus 520 preferably includes at least one port 525 connected to a pump 530 by a conduit 523. The conduit 523 or the breast fixation apparatus 520 preferably includes a valve at port 525. Only the inner chamber need include a reduced pressure environment (or other means for stabilizing the breast).

The fixation apparatus 520 as illustrated is cylindrical, but other shapes (including an at least partially spherical shape) are contemplated as within the scope of the invention. The fixation apparatus 520 preferably includes a substantially rigid plastic portion with an outer surface 521 and an inner surface 522. The plastic (or composite) is preferably substantially transparent to the particular imaging modality (or modalities) used. An internal cavity 524 exists between the inner surface 522 of fixation device and the exterior surface 510 of the breast 500. The cavity 524 is preferably filled with fluid 550. The properties of fluid 550 are identical to previously described fluid 150. The centerline axis of the cylindrical fixation apparatus 520 includes a point 535 that is preferably, but not necessarily, at the center, or substantially overlaps the center, of the tumor mass location 505. However, it should be understood that while such is preferred in a single chamber embodiment, in the dual chamber embodiment the positioning of the inner chamber 520 with respect to the tumor mass location is not of particular importance. The inner chamber 520 should be positioned to provide a reduced pressure environment around the breast 500 (or to otherwise accommodate some other form of means for stabilizing the position of the breast).

With reference to FIG. 6, wherein like elements are labeled as previously described, particle beam 685 is directed at the tumor mass location 505 along a first trajectory. Particle beam 685 is aimed at the center 505 of the spherical portion of outer chamber 620, and the particles are intended to have a penetration depth that extends to that center with a maximum energy dose release in a spherical volume within breast 500. Particles that travel a trajectory normal to the surface of the spherical portion of the outer chamber 620 have an equal path of electron density to the center of the spherical portion. This is because the spherical volume traversed by the particle beam preferably includes either breast tissue or fluid 550. The fluid 550 also at least partially fills the volume between the interior surface 622 of outer chamber 620 and exterior surface 521 of inner chamber 520. The fluid 550 acts as a compensator to allow delivery of spread-out Bragg peak energies precisely to the tumor mass location 505 and to a precise margin of the surrounding tissues. The dual chamber apparatus retains within it the electron-isodense fluid medium and the breast tissue itself. Again, by creating an “anatomy of advantage,” it is possible to deliver uniform dosage—due to substantially identical path radii to the center of the spherical portion.

The fluid 550 within volume 524 of fixation apparatus 520 is preferably held at a slightly reduced pressure to expand the breast 500 to its structural envelope. Air is pumped out of the internal cavity 524 through port 525 fluidly connected by conduit 523 to pump 620. Conduit 523 also passes through a similar port 625 in the wall defining outer chamber 620. Creating the reduced pressure environment requires a substantially fluid tight seal between the rim of the fixation apparatus 520 and the patient's body. As illustrated in FIGS. 5-6, the rim might include patient specific molding 528 that is shaped to conform to the chest of the patient's body, and to provide a fluid tight seal. It should be understood, however, that it is contemplated as within the scope of the invention that the rim of the fixation apparatus may be attached entirely to the chest, entirely to the breast, or to some combination of the chest and breast.

FIG. 6 illustrates how the double chamber embodiment can be rotated around the center of the outer chamber 620 for application of particle beam(s). The center of the at least partially spherical outer chamber 620 at least partially overlaps the center 505 of the tumor mass location. The reduced pressure environment is provided by the inner chamber 520. Adjustments to the outer chamber 620, however, can be made in a more straightforward manner by moving only the outer chamber 620, leaving the patient, and the inner chamber apparatus in contact with the patient, stationary.

With reference to FIG. 6, wherein like reference numerals are used as in FIG. 5, the isocenter of the treatment preferably is at the center of the tumor mass location 505. The breast 500 is placed in a reduced pressure environment within inner chamber 520. The inner chamber 520 and outer chamber 620 are both at least partially filled with a fat equivalent fluid 550. In one application a first particle beam 685 is applied with an isocenter that overlaps the tumor mass location 505. The patient is then rotated (with the beam off), and a second particle beam 695 is applied having an isocenter that overlaps the tumor mass location 505, preferably at the same position as the first particle beam 785. Both the particle beam 685 and the particle beam 695 travel substantially identical path radii (labeled as distance Y in FIG. 6) to the center of the spherical portion of the outer chamber 600. Alternatively, the particle beam could be on the entire time during patient rotation and scan across the surface of the breast while being aimed or directed (having an isocenter) at the tumor mass location 505. The dose will preferably include a tumoricidal sphere around the tumor 505, and a larger sphere of lesser dosage.

It should be understood that relative movement (primarily rotation) between the tumor mass location of the patient laying prone on a treatment platform and the beam preferably occurs by rotation of the patient via translation of the treatment platform. It is contemplated as within the scope of the invention, however, that the patient/breast remains stationary and the breast is beamed from different angles by moving the beam source. Regardless of the mechanism by which the beam's trajectory into the breast and toward the tumor mass location is altered, a preferred implementation of the present invention is a skin-sparing treatment in which the dose to the skin of the breast is spread out. Minimizing of external scarring and skin damage to the breast is considered an important psychological facet to patient treatment. Leaving aside the physiological preference for spreading the dose around the breast to minimize the damage to any given portion of the skin (and underlying tissue between the skin and the tumor mass location), such psychological factors are often of critical importance to patients.

One potential treatment aspect of the present invention entails moving the patient (spinning around the center) during beam-on treatment so that the dose to skin (and other surrounding tissue) is spread out. This geometric dispersion allows for skin-sparing while concentrating the energy via a beam isocenter that overlaps the tumor mass location. Additionally, other treatment aspects of the present invention can be implemented to achieve similar ends. It should be understood that the angles selected for beaming do not require rotation around the full circumference of a circle on the exterior surface of the substantially spherical shape surrounding the breast. Such angles might be selected based on cosmetic effect (i.e. minimize the dosage on the more readily visible top half of the breast when the patent is standing). Thus, in another aspect of the present invention, the patient is not rotated around a circumference, but only around an arc. In a variation of this aspect, even if rotation occurs around an entire circumference, the beam may be turned off during portions of that rotation. For example, the beam might be off when it would otherwise be scanning across the top side of the breast.

In another potential treatment aspect of the present invention, beaming of the breast occurs only along select trajectories, or the beam is only on at select time intervals during scanning of the same around one or more circumferences or arcs on the spherical (including, but not limited to, hemispherical) portion of the chamber enclosing the breast. This is considered desirable as part of an integrated approach to treatment. Namely, some physicians might consider it preferable to avoid beaming that intersects existing wound sites in the form of incisions from past surgical intervention. Thus, in lieu of a more extended period of time in which the patient recovers from such surgical intervention, particle beam therapy might be implemented sooner after surgical intervention. Additionally, more integrated patient therapy might involve planning particle beam therapy to avoid beaming planned future incisions expected to be made in upcoming surgical intervention. Similarly, depending on physician preference, particle beam therapy might be planned in order to beam along or overlap, for example, a biopsy track, on the assumption that cancerous cells might have drifted from the tumor mass location along that track, so that beaming along or overlapping the biopsy track is preferred.

In another embodiment of the present invention the apparatus for treating the breast includes an inner chamber encompassing at least a portion of the breast as illustrated in FIG. 5. The breast is positioned at least partially, and preferably entirely, within an internal volume of the inner chamber. The inner chamber is positioned within an internal volume of an outer chamber. Both the inner chamber and the outer chamber (as illustrated in FIG. 6) are preferably at least partially filled with a fluid having an electron density substantially similar to that of the breast.

Both the inner and outer chambers are preferably, but not necessarily, at least partially spherically shaped. For example, FIG. 1 illustrates a first chamber at least a portion of which has a hemispherical shape. Similarly, FIG. 6 illustrates an outer chamber at least a portion of which has a spherical shape. However, it should be understood that it is contemplated as within the scope of the invention that portions of either or both of the inner and outer chambers might have other shapes. For example, if only radial particle beaming were desired, then either or both of the inner and/or outer chambers might be cylindrically shaped. Those of ordinary skill in the art will understand that it is of greater importance that the shape of the outer chamber be controlled. The shape of the inner chamber is of lesser importance, except as might pertain to shaping the same to insure that it is better secured to the patient. That is to say, insuring equivalent particle path lengths for the particle beam can be controlled by having a substantially spherical (or cylindrical) outer chamber in combination with an inner chamber whose shape need only be determined based on other considerations. Such other considerations include enclosing the breast, being at least partially filled with a fat equivalent fluid, being secured to the patient, and fitting within the outer chamber. In other words, the outer chamber should be positioned so that the tumor mass location is at the center of the spherical portion of the outer chamber. The shape of the inner chamber in a multi-chamber embodiment is not as important to the issue of substantially identical path length of the particle beam (as the beam and breast are shifted relative to one another). Additionally, only portions of the outer chamber need be spherically shaped, those portions preferably comprising the appropriate trajectories for particle beaming. In other words, it is contemplated as within the scope of the invention that only some portion (whether it be an arc, or a circumference of a circle on the outer surface of a sphere) of the outer chamber need include a spherical shape (circular or other shape that has circumferential arcs in a cylindrical embodiment). Additionally, the outer chamber might include more than one spherical portion, and such spherical portions need not be contiguous, though each preferably will have the same center. In yet another embodiment in which multiple tumor mass location “centers” are beamed, the non-contiguous spherical portions of the outer chamber might have different centers.

Furthermore, it should be understood that in the embodiments including multiple chambers, preferably only the inner chamber includes a reduced pressure environment (or other means for stabilizing the breast in space). Thus, the only fluid tight seal that needs to be present (in reduced pressure embodiments) is between the inner chamber and the patient's body (whether to the chest wall, the breast, or some combination of both). Since the patient is preferably lying prone no fluid tight seal is necessary for the outer chamber, the inner chamber with the breast positioned therein simply being dipped into the at least partially fluid filled outer chamber.

Various applications of the present invention were discussed above in conjunction with embodiments of the breast fixation apparatus disclosed in U.S. Pat. No. 6,254,614. However, those of ordinary skill in the art will recognize that the present invention will also find use with other breast fixation apparatus. Various embodiments herein have been described in which stabilization of the breast is accomplished by creating a reduced pressure environment around the breast. Those of ordinary skill in the art will understand that other embodiments of stabilizing the breast are contemplated as within the scope of the invention.

It should further be understood that other forms of stabilizing the breast using tension are contemplated as within the scope of the invention. For example, the inner chamber might be attached (via adhesive, for example) to the breast at a plurality of contact points that might be pulled substantially radially outward to draw the breast outward, that tension fixing the breast in space. Such tension based stabilization of the breast is preferred to fixation through, for example, two or more plates that apply pressure to the breast.

In another example, a self-sucking kind of device could pull the breast into a cup—mold of a hemisphere. Such might be accomplished by fixing the breast using a liquid or gel that solidified into the desired at least partially spherical shape. Various other embodiments along these lines might omit the reduced pressure environment and/or breast fixation apparatus as disclosed in U.S. Pat. No. 6,254,614. Some examples of such embodiments might include plates or other grasping mechanisms for fixing the breast in space. However, the presently preferred embodiment uses a medium surrounding the breast that is fluid and adjustable. As discussed in U.S. Pat. No. 6,254,614 increased pressure might cause cancer cells to pass through the lymph system more rapidly, thus increasing the possibility that the cancer might spread to other portions of the body. Despite such potential issues, it should be understood that pressure based systems for fixation of the breast in space are contemplated as within the scope of the invention. However, such a method for fixation of the breast is less preferred than the reduced pressure environment previously described. As an example, fixation apparatus that involve actual contact with the breast will interpose objects along at least some beam paths to the tumor mass location. The presence of such objects alters the particle path length for those trajectories that intersect such objects. This makes it more difficult to continuously scan along all possible arcs or circumferences of circles of the substantially spherical portion of the chamber that retains therein the fluid having a electron density substantially the same as the breast.

As previously noted, in one embodiment, imaging is preferably done using a combination of CT and 3-D ultrasound. It should be understood that other imaging techniques are also contemplated as within the scope of the invention. Examples of imaging techniques include MRI, CT (including, but not limited to, cone beam CT), 3-D ultrasound, PET scanning, radial and other x-rays, RF targeting with fiducial markers, radio guided imaging (for example, in conjunction with a radioactive seed for localizing the tumor), and other techniques (or combinations thereof) known to those of ordinary skill in the art. Imaging might also be accomplished in conjunction with more invasive procedures, such as via a catheter inserted into the breast with a target for an infra-red imaging system. Such imaging modalities can be registered, integrated and presented for optimal treatment planning.

At least some of the embodiments discussed herein preferably include breast fixation, positioning, verification, and targeting allowing for rotational accuracy in time, speed and duration. Beam energies can be applied in a number of configurations and trajectories, and, as previously mentioned, might be applied to more than one tumor mass location center within the breast as an isocenter. Complete adjustment capability during the entirety of beam transfer is preferred, with multiple safety stops to minimize or prevent patient harm.

The methods and apparatus of the present application are preferably used on patients in a prone position. However, use in a supine or other position is also contemplated as within the scope of the invention. The prone position, however, is more preferred.

Various embodiments of the methods and apparatus of the present invention should allow for low numbers of treatment fractions and, therefore, lower cost and potentially improved cosmetic effect. As one non-limiting example that was previously discussed, the apparatus and method stabilizes the breast and provides equal radial beam distances to the tumor. In essence, the anatomy of the breast is transformed to a near perfect sphere using an electron isodense fluid around the breast with the tumor centrally placed at the center of the spherical portion of the chamber. This arrangement makes treatment planning and execution much simpler. In addition, this device and technique allows for “arc treatment” of the tumor caused by rotating the patient around the isocenter with the beam on—allowing for a spreading of the entrance dose to the skin over a larger area and allowing for higher dosages to be used in a shorter time.

While the invention has been illustrated and described in detail in the drawings and foregoing description, the same is to be considered as illustrative and not restrictive in character, it being understood that only the preferred embodiments have been shown and described and that all changes and modifications that come within the spirit of the inventions are desired to be protected. It should be understood that while the use of words such as preferable, preferably, preferred or more preferred utilized in the description above indicate that the feature so described may be more desirable, it nonetheless may not be necessary and embodiments lacking the same may be contemplated as within the scope of the invention, the scope being defined by the claims that follow. In reading the claims, it is intended that when words such as “a,” “an,” “at least one,” or “at least one portion” are used there is no intention to limit the claim to only one item unless specifically stated to the contrary in the claim. When the language “at least a portion” and/or “a portion” is used the item can include a portion and/or the entire item unless specifically stated to the contrary.

1. A method of treating a tumor mass location in a breast of a patient, comprising:
directing an isocenter of a particle beam at a center of a spherical portion of an outer chamber that overlaps the tumor mass location after passing the particle beam through a first fluid at least partially filling the outer chamber and next passing the beam through a second fluid at least partially filling an inner chamber having a reduced pressure environment that encompasses at least a portion of the breast.

2. The method of claim 1, wherein the particle beam is a proton particle beam, and wherein the first fluid and the second fluid are the same and have an electron density substantially similar to the breast.

3. The method of claim 2, further comprising rotating the particle beam and the breast relative to one another while continuing to direct the isocenter of the particle beam at the center of the spherical portion of the outer chamber.

4. The method of claim 3, further comprising creating the relative rotation by moving a robotic arm connected to a treatment platform upon which the patient lays.

5. The method of claim 3, further comprising deactivating the particle beam at various times while rotating the particle beam and the breast relative to one another.

6. The method of claim 5, wherein particle beaming occurs after a surgical intervention on the breast, and further comprising deactivating the particle beam when the particle beam would otherwise pass through an existing incision site on the breast.

7. The method of claim 5, further comprising deactivating the particle beam when the beam would otherwise pass through a planned future incision site on the breast.

8. The method of claim 3, further comprising directing the particle beam so that it only passes through an underside of the breast of the patient.

9. The method of claim 2, further comprising first imaging the breast and obtaining data regarding the tumor mass location within the breast and providing the data to a computer directing the particle beams at the breast.

10. The method of claim 9, wherein the imaging is done using a combination of 3-D ultrasound and CT.

11. A method of treating a tumor mass location of a breast, comprising:
directing an isocenter of a particle beam toward a center of a spherical portion of a chamber enclosing the breast and through a fluid having an electron density substantially similar to the breast, wherein the center of the spherical portion of the chamber overlaps the tumor mass location, and wherein the particle beam is substantially normal to the spherical portion of the chamber.

12. The method of claim 11, further comprising rotating the breast and spherical portion of the chamber relative to the particle beam to scan through a plurality of particle beam entry angles into the breast while continuing to direct the particle beam substantially normal to the spherical portion of the chamber.

13. The method of claim 12, further comprising directing the particle beam only through an underside of the breast.

14. The method of claim 12, further comprising deactivating the particle beam except when scanning an underside of the breast.

15. The method of claim 12, further comprising rotating the breast and spherical portion of the chamber relative to the particle beam by moving a robotic arm connected to a treatment platform upon which the patient lays.

16. The method of claim 15, further comprising deactivating the particle beam at various times while rotating the particle beam and the breast relative to one another.

17. The method of claim 16, further comprising first imaging the breast and obtaining data regarding the tumor mass location within the breast and providing the data to a computer directing the particle beam at the breast.

18. The method of claim 17, wherein the imaging is done using a combination of 3-D ultrasound and CT.

19. A method of treating a tumor mass location of a breast of a patient, comprising:
reducing pressure within an inner chamber that encloses at least a portion of the breast after creating a fluid tight seal between the inner chamber and the patient;
at least partially filling the inner chamber with a fluid having an electron density substantially similar to the breast;
positioning an outer chamber at least partially filled with the fluid so that a center of a portion of the outer chamber overlaps the tumor mass location;
scanning a particle beam across an arc of the portion of the outer chamber while directing an isocenter of a particle beam at the center of the portion

20. The method of claim 12, further comprising scanning the particle beam only across an underside of the breast.

21. A method of treating a breast cancer tumor mass location, comprising:
stabilizing the breast;
applying an isocenter of a particle beam to a center of spherical portion of an at least partially fluid filled chamber that encloses at least a portion of the breast, wherein the fluid has an electron density substantially similar to the breast, and wherein the center overlaps the tumor mass location.

22. The method of claim 21, wherein the breast is stabilized by providing a reduced pressure environment around the breast.

23. The method of claim 22, further comprising first imaging the breast to determine data regarding the tumor mass location within the breast, and providing the data to a computer controlling the application of the particle beam.

24. A method of treating a tumor mass location in a breast of a patient, comprising:
stabilizing the breast within a reduced pressure environment enclosed by a chamber having a wall at least a portion of which is spherically shaped;
imaging the breast to determine the location of the tumor;
beaming the tumor with particles through the spherically shaped portion of the chamber; and
rotating the beam relative to the tumor mass location around a center of the spherically shaped portion of the chamber, wherein the center overlaps the tumor mass location.

25. The method of claim 24, wherein the patient is prone on a treatment platform, and the patient is rotated with respect to at least one stationary particle beam.

26. The method of claim 24, further comprising directing the particle beam only through an underside of the breast.

27. The method of claim 24, further comprising deactivating the particle beam except when scanning an underside of the breast.

28. The method of claim 24, further comprising using fiducial markers positioned within or on the breast to identify the tumor mass location.

29. The method of claim 24, wherein during stabilization of the breast and during beaming the tumor with particles the chamber is filled with a fluid having an electron density substantially similar to the breast.

30. The method of claim 24, wherein the beaming is controlled to overlap a biopsy track along which prior surgical intervention of the breast has occurred.

31. An apparatus for treating a tumor location in a breast, comprising:
a first wall having an interior surface defining a first chamber sized to encompass at least a portion of the breast;
means for stabilizing the breast within the first chamber;
a second wall having an internal surface defining a second chamber, the second wall including a spherical portion around a center;
wherein the first chamber is smaller than and positioned substantially within the second chamber, and wherein the second chamber is movable with respect to the first chamber to position the center of the spherical portion to overlap the tumor location.

32. The apparatus of claim 31, wherein an edge of the first wall includes an adhesive for attaching the first wall to the breast in a substantially fluid tight fashion.

33. The apparatus of claim 32, wherein the adhesive is a replaceable tape.

33. The apparatus of claim 31, wherein the spherical portion of the second wall is substantially hemispherically shaped.

34. The apparatus of claim 31, wherein at least a portion of the first wall is substantially hemispherically shaped.

35. The apparatus of claim 34, wherein the spherical portion of the second wall is substantially hemispherically shaped.

36. The apparatus of claim 31, wherein at least a portion of the first wall is visually transparent.

37. The apparatus of claim 31, further comprising a fat equivalent fluid at least partially filling the first chamber and the second chamber.

38. The apparatus of claim 37, wherein the means for stabilizing the breast within the first chamber is a reduced pressure environment generated by a pump fluidly connected to the first chamber by a conduit connected to a first port in the first wall, and wherein the conduit passes through a second port in the second wall.

39. The apparatus of claim 37, wherein the first wall defines a first port and further includes a valve within the first port.

40. An apparatus for treating a tumor location in a breast, comprising:
a first shell having an interior surface defining a first chamber sized to encompass at least a portion of the breast, wherein at least a portion of the shell defines a spherical portion having a center, and wherein at least a portion of the first chamber is filled with a fluid having an electron density substantially similar to breast tissue; and,
means for stabilizing the breast within the first shell.

41. The apparatus of claim 40, wherein the portion of the first shell is substantially hemispherically shaped.

42. The apparatus of claim 41, wherein an edge of the shell includes an adhesive for attaching the shell to the breast in a substantially fluid tight fashion.

43. The apparatus of claim 42, wherein the adhesive is a replaceable tape.

44. The apparatus of claim 40, where an edge of the shell includes molding shaped to overlay an area around the breast.

45. The apparatus of claim 40, further comprising a second shell having an internal surface defining a second chamber, the second shell including a spherical portion around a second center, wherein the first chamber is smaller than and positioned substantially within the second chamber, and wherein the second chamber is movable with respect to the first chamber to position the second center to overlap the tumor location.

46. The apparatus of claim 45, further comprising the fluid at least partially filling the second chamber.

47. The apparatus of claim 46, wherein the means for stabilizing the breast within the first chamber is a reduced pressure environment generated by a pump fluidly connected to the first chamber by a conduit connected to a first port in the first shell, and wherein the conduit passes through a second port in the second shell.

48. The apparatus of claim 46, wherein the first shell defines a port.

49. The apparatus of claim 48, further comprising a valve in the port of the first shell.
